(19) United States
(12) Patent Application Publication
(10) Pub. No.: US 2003/0153978 A1
(43) Pub. Date: Aug. 14, 2003

(54) Apparatus and method of ligament balancing and component fit check in total knee arthroplasty

(75) Inventor: Leo A. Whiteside, Chesterfield, MO (US)

Correspondence Address: POLSTER, LIEDER, WOODRUFF & LUCCHESI, 763 SOUTH NEW BALLAS ROAD, ST. LOUIS, MO, 63141-8750, US

(73) Assignee: Whiteside Biomechanics, Inc.

(21) Appl. No.: 10/072,372

(22) Filed: Feb. 8, 2002

(51) Int. Cl. A61F 2/38; A61F 2/46

(52) U.S. Cl. 623/20.21; 606/102; 623/914; 73/172

(57) ABSTRACT

Apparatus and method for determining the kinematics of a large joint (e.g., a knee) undergoing arthroplasty, for comparing the kinematics of the joint undergoing surgery with the kinematics of the normal joint, and for providing the surgeon with information allowing the balancing of the ligaments of the joint upon the installation of a prosthesis replacement joint. The apparatus includes optical targets mounted on joint replacement components fitted to the resected bones of the joint. One or more video cameras are used to obtain a series of images of the various optical targets as the extremity is flexed, extended, and rotated. A computer is responsive to the images of the targets to determine the kinematics of the joint as it is flexed, extended and rotated. The computer compares the kinematics of the observed joint with the kinematics of a normal joint and determines, based on anomalies of the observed joint relative to the normal joint, whether other implant components or spacers are required, and determines which ligaments must be relaxed or contracted so that the ligaments are balanced. The computer displays suggestions to the surgeon for changing components and for relaxing or contracting specific ligaments such that the ligaments of the joint is balanced with the prosthesis joint in place. A method of computer ligament balancing is also disclosed.

### CROSS REFERENCE TO RELATED APPLICATIONS

### STATEMENT REGARDING FEDERALLY SPONSORED RESEARCH OR DEVELOPMENT

### BACKGROUND OF THE INVENTION

It is oftentimes necessary to replace a patient's joint (e.g., a knee) because of chronic disease, such as arthritis or other degenerative bone or joint diseases. Over time, as such disease conditions progress, the bones forming the joint become deformed thus causing the ligaments of the joint to stretch or contract so as to accommodate to the deformed joint. Upon surgical replacement of the deformed joint with a prosthesis joint, the latter will be of proper size, position, and orientation with respect to size of the patient and with respect to the patient's anatomical landmarks and reference axes. In such joint replacement surgery, it is desirable that the ligaments of the joint remain in tact and remain attached to the bones immediately above and below the joint being replaced. However, because the ligaments have been stretched or contracted by the diseased joint, the ligaments oftentimes will not hold the prosthesis joint components in proper relation to one another as the joint is flexed and extended. This may result in improper kinematics for the joint which will impair the mobility of the patient and may result in excessive wear of the joint components.

It is conventional in total knee arthroplasty for the surgeon, after the femoral and tibial bone cuts have been made, to fit trial implant components in the knee and then to flex, extend, and rotate the knee in order to assess varus, valgus, rotational motion, and the anterior and posterior stability of the knee. This information is conventionally used by the surgeon, based on the surgeon's subjective judgement, to determine which ligaments of the knee need to be released or contracted in order to insure that optimal knee function will result from the joint replacement surgery. Because the knee is complex and its behavior is difficult to accurately assess, the balancing of the ligaments has been difficult for surgeons.

Heretofore, apparatus were known that aided the surgeon in determining the position of the bones of the joint and to aid in positioning the bone cuts to be made so that the prosthesis joint components will properly fit the bone cuts and so that the prosthesis joint is properly position with respect to the bones of the joint. Such prior art alignment and bone resection instruments are shown in my prior U.S. Pat. Nos. 4,467,801, 4,474,177, 4,721,104, 4,722,330, 4,731,086, 4,935,023, 5,002,545, 5,098,436, 5,019,104, 5,100,409, 5,176,684, 5,275,603, 5,342,367, 5,415,662, 5,431,656, 5,578,039, 5,609,642, 5,628,749, 5,667,511, 5,683,469, 5,683,470, and 6,683,397, which are herein incorporated by reference.

Reference may also be made to International Patent Application WO 98/40037 which discloses a system utilizing bone indicators having light emitting diodes (LEDs) fixed thereto which are viewed by stereoscopic video cameras such that a computer responsive to cameras may determine the position of the bone markers and thus the position of the bones relative to one another. This information is then utilized to determine the location of the bone cuts to be made in the proximal end of the tibia and the distal end of the femur for proper placement of the prosthesis joint components.

Further, reference may be made to U.S. Pat. No. 5,249,581 which discloses a bone alignment system that utilizes bone markers affixed to the bones of the extremity with the bone markers being viewed by stereoscopic video cameras to determine the position of the bones relative to one another.

In a paper entitled “Visualization of the Articulation of Replaced Knee Join Surfaces After Total Knee Arthroplasty”, by Toyohiko Hayashi et al. of the Graduate School of Science and Technology, Niigata University (1997), an intraoperative 6-degree of freedom measurement system and three dimensional analysis of knee movement was disclosed in which stereoscopic video cameras using charge coupled devices (CCD) were used to determine the position of light emitting diodes attached to the femoral and tibial component by means of a measurement bow. This system determined the relative movement of femoral and tibial components such the relative movement of the joint surfaces could be qualitatively assessed.

A similar system is disclosed in an article published in the Frontiers Med. Biol. Engng., Vol. 9, No. 4, pp 262-273 (1999) by Katsutoshi Nishino et al. A similar system was disclosed in a paper entitled “Intra-Operative Monitoring of Knee Motion of Total Knee Arthroplasty Using 6 DOF Photostereometric System, KKN/1B”, by Hayshi, T. et al. presented at the 44^{th} Annual Meeting, Orthopaedic Research Society (1998).

However, the prior art did not include a system or method that aided the surgeon during the surgery to evaluate the ligaments of the joint and that aided or guided the surgeon in balancing the ligaments of the joint. Further, the prior art bone positioning systems did not utilize trial prosthesis joint components to determine the kinematics of the joint such that while the knee was being manipulated by the surgeon in extension, flexion and rotation, the kinematics of the trial components relative to one another could be objectively evaluated in order to assess varus, valgus, rotational motion, and the anterior and posterior stability of the knee and so as to check the fit and size of the replacement joint components.

### SUMMARY OF THE INVENTION

This invention relates to a system and method of balancing ligaments in large joint replacement surgery, and is more particularly related to a system and method of ligament balancing in total knee arthroplasty. More specifically, this system and method utilizes a plurality of optical targets (either passive or active) affixed to joint replacement trial components fitted to the resected bone surfaces and viewed by a video camera such that as the surgeon manipulates the patient's joint in extension, flexion, and rotation, a computer coupled to the video camera determines the position and orientation of the trial components relative to one another. In this manner, the computer may compare the kinematics of the trial components as they move relative to one another as the joint is flexed and extended and the known nominal kinematics for those joint components in a normal knee such that the computer can determine which ligaments of the joint are overly tight and which are lax. Thus, the computer may suggest to the surgeon which ligaments of the joint need to be corrected so as to result in the ligaments being balanced with the prosthesis joint installed. Further, the computer may perform a fit check of the prosthesis joint relative to the patient and determine if other joint components are more appropriate.

Among the several objects and features of the present invention may be noted the provision of system which aids the surgeon in identifying which ligaments need to be released or contracted in order for the knee to function normally;

The provision of such a system which compares the kinematics of the resected knee with trial prosthesis joint components fitted to the resected femur and tibia upon flexion, extension, and rotation to the normal knee and which generates a surgical plan based on the correct alignment throughout the arc of flexion for ligament release or contracture based on the function of each ligament;

The provision of such a system which results in optimal knee function including correct valgus-varus alignment in all positions of flexion;

The provision of such a system which tests the tension of the ligaments of the knee in different positions of knee flexion, compares the tension of the ligaments with the tension of the ligaments of the normal knee, and determines which ligaments must be released in order for the knee to function normally while leaving those ligaments functioning normally;

The provision of such a system which guides the surgeon in carrying out surgical procedures for relaxing identified ligaments such that upon completion of the surgery the deformed ligaments are balanced about the prosthesis joint thus facilitating a more rapid recovery for the patient and promoting a more satisfactory outcome for the surgery;

The provision of such a system which minimizes instability and which minimizes wear of the prosthesis components;

The provision of such a system and method which may also be used to check the fit and the function of trial components fitted to the resected bones of the joint so as to insure proper fit and function of the prosthesis joint; and

The provision of a computer-aided method of aiding the surgeon in so balancing the ligaments which is easy to use, which is sufficiently accurate such that the surgeon can base his or her procedure on the suggestions made to the surgeon by the computer, which is reliable in operation, and which is of reasonable cost.

Other objects and features of the system and method of this invention will be in part apparent and in part pointed out hereinafter.

Briefly stated, the system and method of the present invention comprises a computer-aided system in which one or more digital video cameras view targets on trial prosthesis joint components fitted to the resected knee as the knee is flexed, extended and rotated to determine the kinematics of the knee with the prosthesis components in place and with the ligaments unbalanced. The kinematics of the knee prior to balancing are compared to the kinematics of the normal knee and abnormalities due to ligament unbalance are determined. A computer then identifies the ligaments of the knee which must be balanced or released so that the knee will function normally. The computer then guides the surgeon to release or contract such identified ligaments.

Apparatus of the present invention for balancing the ligaments of a large joint during joint replacement surgery is disclosed. During knee replacement surgery, the apparatus of this invention compares the ligaments of the joint undergoing surgery with the ligaments of a normal joint, and provides the surgeon with information allowing the balancing of the ligaments of the joint after the prosthesis joint has been installed. Specifically, the apparatus comprises a plurality optical targets mounted on the prosthesis joint fitted to the resected bone surfaces of the joint or otherwise in known positions with respect to the bones of the joint. One or more video cameras obtain a series of images of the optical targets as the joint is flexed, extended, and rotated. A computer responsive to the images of the targets determines the kinematics of the prosthesis joint as it is flexed, extended and rotated. The computer compares the kinematics of the prosthesis joint with the kinematics of a normal joint and determines, based on anomalies of the observed prosthesis joint relative to the normal joint, whether other prosthesis joint components or spacers are required in order for the prosthesis joint to operate properly and determines which ligaments of the joint must be relaxed or contracted so that the ligaments of the joint are balanced after installation of the prosthesis joint. The computer displays suggestions to the surgeon for changing the prosthesis components and/or for adding or removing the spacers, and displays suggestions for relaxing or contracting specific ligaments such that the ligaments of the joint are balanced with the replacement joint in place.

Stated differently, apparatus of the present invention determines the kinematics of a large joint undergoing arthroplasty, compares the kinematics of the joint with trial prosthetic joint components fitted to resected bone surfaces of the joint with the kinematics of a normal joint, and provides the surgeon performing the surgery with information allowing the balancing of the ligaments of the joint with the prosthesis joint in place. The apparatus includes a plurality of optical targets mounted on the trial joint replacement components fitted to the resected bone surfaces of the joint with the position and orientation of each of the targets being known with respect to their respective trial joint replacement components such that by knowing the position and orientation of the targets of the respective trial joint replacement component the position and orientation of the trial joint replacement components is known. The system includes one or more video cameras with the cameras being positioned with respect to the joint such that as the joint is flexed, extended, and rotated by the surgeon, the targets are within the field of view of the cameras, the cameras obtaining a series of images of the optical targets as the joint is flexed, extended, and rotated, a computer responsive to the images of the targets for determines the position and orientation of the targets and hence of the trial joint replacement component with respect to the cameras. The computer determines the position and orientation of the trial joint replacement components with respect to one another in each of the positions so as to determine the kinematics of the joint as it is flexed, extended and rotated. The computer compares the kinematics of the observed joint with the kinematics of a normal joint and determines, based on anomalies of the observed joint relative to the normal joint, whether other implant components or spacers are required, and determines which ligaments must be relaxed or contracted so that with the joint components installed the ligaments are balanced. The computer displays suggestions to the surgeon for changing components and for relaxing or contracting specific ligaments so that the ligaments may be balanced.

Further, apparatus of the present invention determines the kinematics of prosthesis joint components relative to one another and relative to a normal joint during joint replacement surgery to aid the surgeon in fitting joint components to the resected bones of the joint undergoing surgery and aids the surgeon in balancing the ligaments of the joint after a prosthesis joint has been installed. The apparatus comprises a first set of optical targets mounted to a first trial joint component fitted to a first resected bone of the joint. The first set of optical targets is in a predetermined geometrical relationship with respect to the first trial joint component. A second set of optical targets is mounted to a second trial joint component fitted to a second resected bone of the joint. The second set of optical targets is in a predetermined geometrical position with respect to the second trial joint component. At least one video camera is disposed to receive images of the first and second sets optical targets. This at least one video camera has a field of view and the first and second sets of optical targets are within the field of view as the joint is flexed, extended and rotated. A computer is operatively connected to the at least one camera. The computer is responsive to the images of the first and second sets of targets to determine values of the position and orientation of the trial joint replacement components in space and with respect to one another and determines the kinematics of the trial joint components relative to one another as the joint is extended, flexed and rotated. The computer displays information to the surgeon relating to the balancing of the ligaments of the joint.

This invention further comprises a method of balancing the ligaments of the knee during total knee arthroplasty. The method comprises resecting the distal end of the femur so as to receive a femoral prosthesis implant and resecting the proximal end of the tibia so as to receive a tibial prosthesis implant. Then, trial implant components are fitted the resected ends of the femur and tibia. The trial components each have a plurality of optical targets affixed thereto with the position and orientation of the targets with respect to its respective trial component is known. The surgeon then manipulates the extremity in flexion, extension and rotation. A video camera views the optical targets on the trial components as the extremity is manipulated. A computer responsive to the images generated by the camera determines the position and orientation of the targets with respect to the camera. The computer further determines the position and orientation of the trial components with respect to one another and the kinematics of the trial components relative to one another. The computer identifies anomalies between the observed kinematics of the trial components and the known kinematics for the trial components in a normal knee which anomalies result from an unbalance of the ligaments of the knee. The computer suggests to the surgeon which of the ligaments of the knee should be adjusted either by relaxing or contracting such ligaments so that upon installation of the prosthesis components, the ligaments of the knee are balanced.

### BRIEF DESCRIPTION OF THE DRAWINGS

FIG. 1 is a perspective view of an operating room in which the system of the present invention is being used in a total knee arthroplasty surgical procedure where a patient rests on an operating table with the patient's knee undergoing surgery in a flexed position and having joint replacement trial components installed on resected bone surfaces with the trial pieces having a plurality of optical targets affixed thereto, a video camera supported on a movable post so the camera may be positioned to view the targets while a surgeon manipulates the extremity assess varus, valgus, rotational motion, and the anterior and posterior stability of the knee, and a computer connected to the camera for determining the kinematics of the trial pieces as the extremity is manipulated and for suggesting to the surgeon a procedure for balancing the ligaments;

FIGS. 2A and 2B are perspective views of the patient's knee having trial components fitted to the resected bone surfaces with the trial components having optical targets thereon which are viewed by the camera as the surgeon manipulates the extremity to assess varus, valgus, rotational motion, and the anterior and posterior stability of the knee;

FIG. 3 is a perspective view of a surgeon using a surgical instrument to balance ligaments where the instrument has targets thereon such that the system of this invention can determine the amount of adjustment that has been performed by the surgeon and how much more adjustment is required before the ligaments are balanced;

FIG. 4 is a side elevational view of the medial side of the right knee in its flexed position illustrating the posterior portion of the medial collateral ligament which is loose in flexion and the anterior portion of the medial collateral ligament which is tight and which contributes to ligament imbalance;

FIG. 5 is a view similar to FIG. 4 in which the lower leg is extended illustrating that the posterior portion of the medial collateral ligament is tight in extension and the anterior portion of the medial collateral ligament slackens so that the knee with the trial pieces in place on the resected surfaces of the femur and the tibia are substantially balanced;

FIG. 6 is a view generally similar to FIG. 4 further illustrating the release of the anterior fibers of the medial collateral ligament by the surgeon using an instrument in accordance with this invention having targets thereon so that the video camera can track the position of the instrument and so that the system of this invention can determine the amount the surgeon has balanced the medial collateral ligament and how much more surgical adjustment is needed to achieve ligament balance;

FIG. 7 is a view similar to FIG. 5 illustrating that the anterior fibers of the medial collateral ligament have been surgically released and that the medial stability of the knee in extension has been substantially restored because the posterior portion of the medial collateral ligament and the posterior medial capsule function substantially normally; and

FIG. 8 is a view similar to FIG. 6, except the pes anserinus has been omitted for clarity, illustrating that in flexion, the anterior portion of the medial collateral ligament is no longer tight and the posteromedial oblique portion of the medial collateral ligament now acts as a secondary medial stabilizer in flexion.

Corresponding reference characters indicate corresponding parts throughout the various drawings.

### DESCRIPTION OF PREFERRED EMBODIMENTS

This invention relates to a computer-aided system and method of determining ligament unbalance in a large joint (e.g., knee) during joint replacement surgery and guiding a surgeon to balance the ligaments so that the joint and the prosthesis joint will function normally. This system is also useful for determining the position and orientation of trial joint prosthesis components fitted to the resected surfaces of the joint bones such that the size and fit of the prosthesis components may be assessed and whether joint components of a different size should be fitted or whether spaces should be utilized in conjunction with the joint components. While this invention is described in relation to total knee arthroplasty, those skilled in the art will recognize that the system and method described herein can be applied in other joint replacement surgery, such as hip, ankle and shoulder surgery.

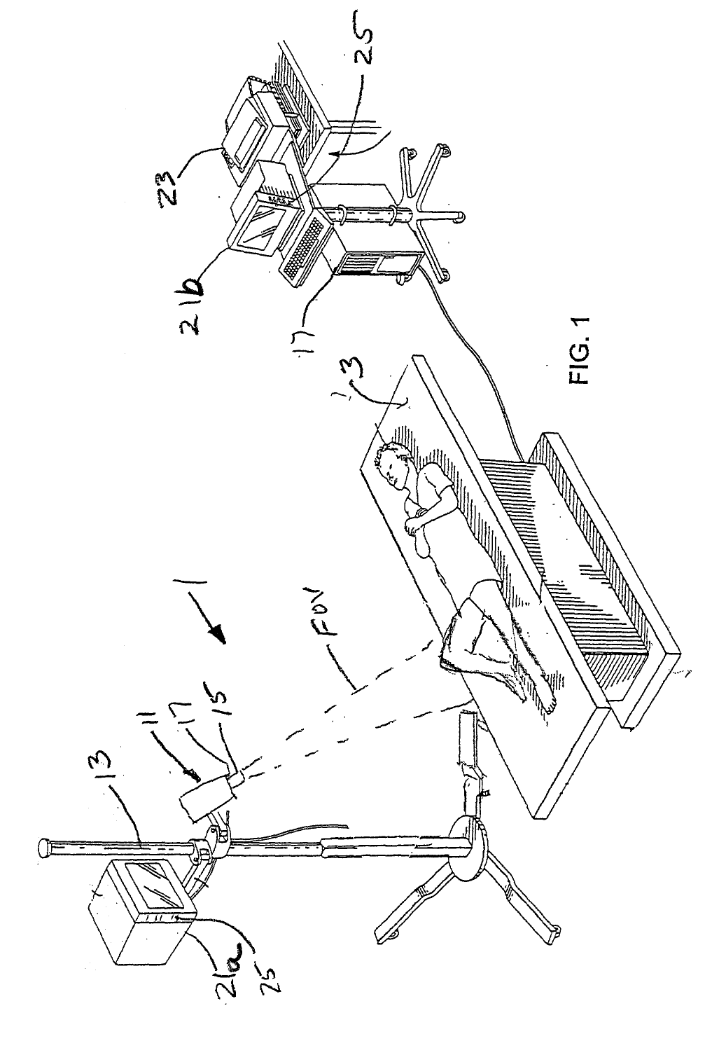

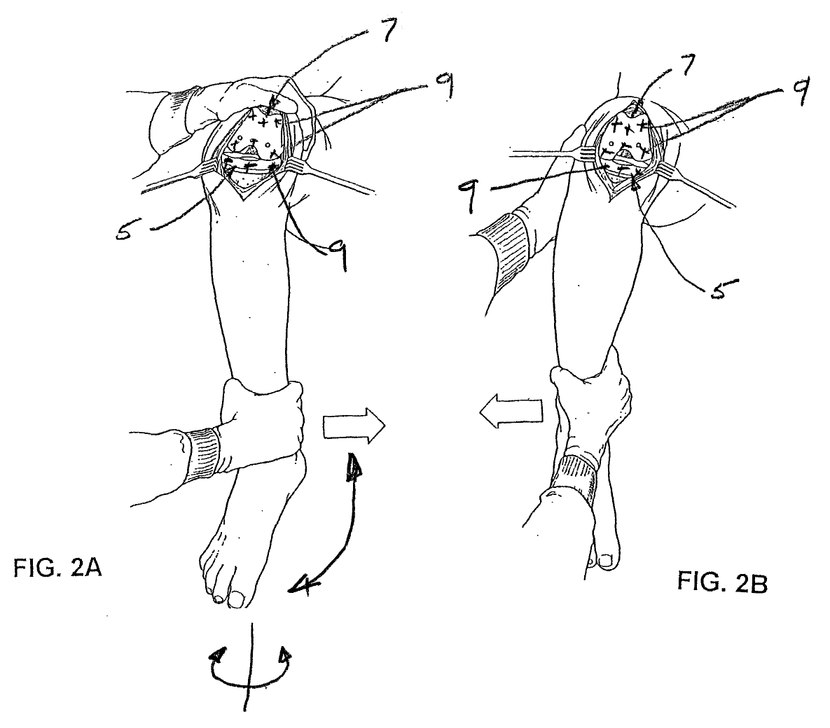

Referring now to FIG. 1, apparatus of the present invention is indicated in its entirety at 1. As shown, a patient P undergoing joint replacement surgery is laid on a conventional operating table 3 and the extremity (e.g., the patient's right leg) is supported in the conventional manner. As shown, the surgery has progressed to the point where trial prosthesis joint components have been fitted to the resected bone surfaces of the joint. As shown in FIG. 2, a tibial trial prosthesis component 5 has been fitted to the resected bone surface of tibia T and a femoral trial prosthesis component 7 has been fitted to the resected bone surface of femur F. These trial pieces 5 and 7 are similar in size, shape and kinematic function to the actual prosthesis joint components that will be implanted, but are only temporarily fitted to the resected bone surfaces to check the fit and function of the joint and to assess varus, valgus, rotational motion, and the anterior and posterior stability of the knee during the surgery. As is conventional, if a problem is noted during this fit check, certain procedures are available to improve the fit and function of the replacement joint. For example, the trial pieces may be exchanged for trial pieces of other sizes and shapes, or spacers can be added or removed. Then, if the fit of the trial pieces is satisfactory, the trial pieces are removed and the permanent prosthesis components are fitted and permanently secured in place in the conventional manner.

In accordance with the system and method of this invention, trial pieces 5 and 7 are each provided with a plurality of optical targets 9 affixed to each respective trial piece in positions and orientations that are accurately known with respect to the trial piece. A sufficient number of targets 9 are affixed to the trial pieces such that the position and orientation of the trial pieces may be determined as the surgeon manipulates the leg, as hereinafter described. Preferably, four or more of such targets 9 on each of the trial pieces should be within the field of view of the camera as the leg is manipulated. The position and orientation of the targets 9 with respect to its trial piece is known such that articulating (condyle) surfaces of the joint component relative to the targets is known in three dimensions. These targets may be small retro-reflectors which reflect incident light back to the light source emitting the energy illuminating the targets. Alternatively, the targets may be active devices, such as light emitting diodes (LEDs), that emit substantially a point source of light. It will be appreciated by accurately knowing the position and orientation of four (4) or more of such targets on one of the trial components, the spatial position and orientation of the trial piece may be determined.

As used in this disclosure, the term “position and orientation” is defined to mean that the position in space of an object, such as the trial pieces 5 and 7, is known with respect to all six (6) degrees of freedom relative to a known coordinate system. Because the trial pieces are solid members and because the position and locations of targets 9 on the trial are fixed, by knowing the position and orientation of the targets in space, the position and orientation of all surfaces of the trial pieces is also known. Of course, if the position and orientation of both trial pieces is known with respect to a single reference system, the position and orientation of the trial pieces relative to one another may be determined.

Further in accordance with the system of this invention, a video camera 11 is supported on an elevated post 13 which may be moved to a position where the camera may observe the joint undergoing surgery while the surgeon manipulates the patient's joint in flexion, extension and rotation to assess joint function and the tightness or laxness of the patient's ligaments. Preferably, camera 11 is a digital video camera having a lens 15 which focuses an image on a charge coupled device (CCD), not shown, within the camera. As is well known, such a charge coupled device is relatively small in size (e.g., a fraction of an inch on each side) and has a flat surface on which a large number of photosensitive elements, referred to as pixels, are arranged in an array of rows and columns. Each of the pixels generates a small voltage or other electrical signal proportional to the amount of light impinging on the pixel. In the well known manner, the output of these pixels is then processed by the camera to form an image. Such cameras are capable of generating a color image, but for purposes here, the camera need only generate a black and white image with multiple shades of gray (e.g., 256 shades of gray). However, within the broader aspects of this invention, color images are usable. Lens 15 has a field of view FOV which is sufficiently wide as to include the trial pieces 5 and 7 as the surgeon manipulates the patient's leg during surgery to evaluate the trial components.

As indicated at 17, a computer is responsive to the images of the targets 9 of trial pieces 5 and 7 generated by camera 11 to determine the position and orientation of trial pieces relative to camera 11 and relative to one another. In this manner, by analyzing a series of images of the trial pieces, the kinematics of the trial pieces relative to one another may be determined as the joint is manipulated by the surgeon during the surgery in flexion, extension, and rotation. Thus, the actual kinematics of the joint components, as determined by camera 11, can be compared by computer 17 to the known kinematics of the trial components and the computer may determine any anomalies between the observed and known kinematics. The computer is programmed to identify such anomalies and to ascertain whether such anomalies are due to one or more of the trial components being of the wrong size or fit for the patient and/or whether such anomalies are due to an imbalance of the joint ligaments. Further, computer 17 will provide suggestions to the surgeon regarding trying different sizes of trial components, and as to whether the use of shims or spacers should be used with the trial pieces. Then, once the trial pieces of proper size are fitted to the resected bone surfaces, further manipulation of the extremity will determine whether the ligaments of the joint are balanced and, if not, the computer will display suggestions to the surgeon as to which ligaments should be released or contracted so that the resulting ligament structure for the joint with the proper prosthesis joint in place will be balanced.

The anatomical relationship of the ligaments of the knee are generally repeatable from patient to patient with the main differences being the result of the diseased or deformed joint that is being surgically replaced. Thus, even though the ligaments may have been stretched or contracted by the deformed joint, the attachment of the various ligaments relative to the femur, tibia and fibula are in substantially the same position as in a normal joint. Thus, computer 17 may be provided with a three dimensional model of the ligament structure of a normal joint. As the computer detects the above-noted anomalies of the ligaments of the joints, such anomalies relative to the ligaments of a normal joint may be displayed on the monitor so that the surgeon can visually see the condition of the ligaments that the computer observed and so that the ligaments identified to be relaxed or tightened may be correlated with the ligaments of the patient. In addition, the computer may display the amount the identified ligaments should be adjusted.

Still further, the system of this invention may observe the surgeon's use of a surgical instrument, such as an oestome, used to relax a ligament to determine whether the ligament has, for example, been sufficiently released. Computer 17 may thus provide instructions or suggestions to the surgeon as to whether the ligaments have been sufficiently relaxed or otherwise corrected.

If targets 9 are passive retro-reflectors, it is desirable that they be illuminated with light of a predetermined wavelength such that the targets may be more readily acquired within the image of the trial pieces, as seen in the image of camera 11 and as processed by computer 17. It will be appreciated that many items besides the targets will be present within the image and that the image will change from second to second because of changes within the field of view of the camera. In order to aid the computer to identify (i.e., acquire) the targets (and hence the trial pieces 5 and 7), it may be preferred to surround lens 15 of camera 11 with an array of light emitting diodes (LEDs) 19 which emit light of a known wavelength along an axis generally parallel to the viewing axis of the lens. Because the retro-reflector targets will reflect light impinging on them in substantially the opposite direction that the light was received, a high proportion of the light will be reflected back to the lens 15 along the axis of the lens. Filters can them be used to eliminate substantially all other objects in the image except the images of the targets. In manners well known to those skilled in the “machine vision” art, various target acquisition techniques are known to insure that the images or the targets appearing in the image are in fact images of the targets. It is also known that the images of the targets on one of the trial pieces 5 or 7 can be discriminated from one another. In this manner, robust images of both of the trial pieces 5 and 7 will be generated by the camera as the joint is articulated and these images will provide time coordinated information as to the position and orientation of both of the trial pieces at the same instant in time. This information can then be processed by computer 17 to determine the kinematics of the trial pieces as the knee is flexed, extended, and rotated.

As noted, the position and orientation of targets 9 on trial pieces 5 and 7 are accurately known. However, it is important to be able to determine with sufficient accuracy the actual center of the targets appearing in the image such that the actual position and orientation of the targets on their respective trial pieces 5 and 7 may be determined so that the spatial orientation and position of the trial pieces with respect to the camera (and hence with respect to one another) may be accurately ascertained. If retro-reflectors are used, it will be appreciated that while the physical targets 11 may be quite small (e.g., only a few millimeters in diameter). However, the image of the targets will encompass a relatively large number of pixels on the image. In addition, the image of a target may only partially illuminate pixels on the edges of the target image. Edge detection methods are well know to those skilled in the “machine vision” arts which allow a determination of which pixels are within the image of the target and which pixels are outside of the image of the target, and which pixels are partially illuminated and should be considered as part of the image. Still further, various methods are known by which the center or centroid of the image of the target can be determined. In this manner the position of each target 9 in the image can be accurately ascertained.

Once the positions of targets 9 on the trial pieces 5 and 7 in the image have been accurately ascertained, algorithms are known which allow computer 17 to determine the spatial position and orientation of each of the trial pieces 5 and 7 with respect to the camera from the two dimensional images of the targets. Essentially, these algorithms observe the relative position of the targets 9 within the image and then, from this information, deduce the position and orientation of the trial pieces 5 and 7 that would result in the observed image as viewed by the camera. As exemplified by the above-noted vision prior art wheel aligner patents, such algorithms are capable of determining the position and orientation of the trial pieces relative to the camera to within a small fraction of a millimeter (e.g., to within about ¼ mm.) and to within a small fraction of a degree (e.g., to within about 1/100^{th} of a degree). Such accuracy is more than sufficient in the instant application to determine the position and orientation of the trial pieces 5 and 7 to assess the fit of the trial pieces, to permit the determination of the kinematics of the trial pieces relative to one another as the joint is manipulated, and to assess the condition (i.e., the imbalance) of the ligaments with the trial pieces installed such that the computer 17 can suggest a procedure for balancing the ligaments with the prosthesis joint installed.

While the use of a single camera is preferred because of its lower cost, it will be understood that within the broader aspect of this invention, the use of multiple cameras, such as shown in U.S. Pat. No. 5,249,581, may be employed. It will also be appreciated that the use of passive retro-reflector targets 9 is preferred because of their simplicity and relatively low cost and because the trial pieces can be readily autoclaved. However, as disclosed in the above-noted U.S. Pat. No. 5,249,581, active light emitting diode targets could be employed.

It will also be understood that the term “target” as used herein refers to some indicia associated with the trial pieces 5 and 7 that allows the position and orientation trial pieces to be determined. In place of targets 9, a laser could be used to “paint” an array of laser lines on the trial pieces with the spacing and arrangement of the laser lines being known such that these laser lines function as a target. Then, by camera 11 observing the pattern of the known laser lines on the target, the position and orientation of the target to yield such a pattern of laser lines can be determined by computer 17. Also, actual indicia (i.e., scribe lines) can be placed on the trial pieces with the position, width, and spacing of such trial lines relative to the trial pieces being known. By the camera observing which of these trial lines are visible as the joint is flexed, the relative position and orientation of the trial pieces 5 and 7 can be determined. Thus, the term “target”as used herein encompasses both active and passive targets, laser lines, scribe lines, and other indicia, as above described.

Examples of algorithms that determine the position and orientation of targets 9 with respect to the camera 11 are well known in other applications. For example, reference may be made to the algorithms disclosed in such U.S. Pat. Nos. as 5,675,515, 5,724,743, 6,134,792, and 6,252,973. Additionally, reference may be made to a paper by Tsai et al, entitled “A Versatile Camera Calibration Technique for High-Accuracy 3D Machine Vision Metrology Using Off-The-Shelf TV Cameras and Lenses”, August 1987, vol. RA-3, No. 4, pp. 323-344 in IEEE Journal of Robotics and Automation. The last-mentioned patents and the Tsai et al. paper are herein incorporated by reference. While the above-noted patents describe systems for determining the alignment of automotive wheels, those skilled in the art will recognize that the methodologies and algorithms disclosed therein may be readily modified and employed for the determination of the position and orientation of trial pieces 5 and 7 with respect to camera 11 in much the same manner as the targets affixed to the wheels of a vehicle may be determined with respect to the camera. It will be appreciated that an iterative algorithm may be preferred to determine the position of targets 9 within the image. Of course, once the locations of targets 11 for the trial pieces are determined within the image, it is a relatively straightforward (albeit somewhat complex) mathematical procedure to transform the position and orientation of each of the trial pieces relative to the camera into a reference system in which the position and orientation of the trial pieces relative to one another is known. This of course allows the position of the trial pieces relative to one another to be determined as the joint is articulated in a series of images such that the kinematics of the prosthesis joint can be determined.

It will be understood that camera 11 generates many images each second, depending on the scan rate of the camera. The number of these images that can be processed by computer 17 each second depends on the speed of the computer to complete the require calculations, the number of targets visible on the trial pieces 5 and 7, and other factors. However, it will be understood that for the purposes of this invention, it not necessary to compute the position and orientation of the trial pieces for each image generated by the camera, but rather only a relatively few images of the trial pieces need be calculated in order to determine the kinematics of the trial pieces with respect to one another as the knee is flexed, extended and rotated. In this manner, a series of progressive images of the trial pieces 5 and 7 may be calculated showing the position of one trial piece relative to the other in three dimensions. This series of images of the trial pieces thus constitutes the kinematics of the trial pieces, as installed in the patient. Economical cameras and computers are readily commercially available that are capable of determining the position and orientation of both trial pieces 5 and 7 three to ten times each second. Because the rate of movement of the joint by the surgeon is relatively slow, the computer is able to display a substantially real time calculation of joint position.

Computer 17 may be a so-called IBM compatible personal computer having a state of the art Intel Pentium III or better processor, 256 meg of RAM, a disk drive of 10 or more gigabytes, and the usual input/outputs, sound cards, video cards and the like. One or more video monitors 21 may be driven by the computer. A first monitor 21a,may be supported on post 13 proximate camera 11 so that it may be viewed by the surgeon during the surgery. Another monitor 21b may be positioned near computer 17. These monitors are used to display information useful to the surgeon for aiding in fitting of the trial pieces 5 and 7 and in aiding the balancing of the ligaments. The monitors may also display the actual image being viewed by camera 11 to insure that the targets 9 of the trial pieces are within the field of view FOV of camera 11 prior to and while the surgeon manipulates the joint. Further, computer 17 may be provided with a printer 23 and with audio speakers 25 which may be built into the monitors 21. The printer may be used to print out a plan to the surgeon of how best to balance the ligaments, showing the surgeon which ligaments should be contracted and which should be released. The printer may also print out a plan for adjusting the trial pieces for insuring a better fit. The speakers 25 may provide aural instructions or suggestions to the surgeon.

Video camera 11 may be anyone of a number of relatively inexpensive video cameras commercially available from a number of different companies. It is not necessary that the camera be a high resolution camera. It will also be recognized, that it may be desirable that the camera be specially constructed for use with the apparatus of this invention so that the camera will be especially adapted for this application. It will be understood that such cameras specially constructed for use with the apparatus of this invention may be tailored to have only the features needed for this application and expensive features, such as zoom lens and the like may be eliminated, thus making the camera even more economical.

As used in this disclosure, the term “kinematics” means the pattern of motion having six degrees of freedom of one of the trial pieces relative to the other as the trial pieces 5 and 7 are installed on the resected bone surfaces of the knee and as the knee is articulated and rotated. It is understood that the femoral trial piece 7 has surfaces thereon that simulate the condyles of the femur and that the tibial trial piece 5 has surfaces thereon which simulate condyles of the tibia so that upon articulating the knee with the trial pieces in place, the kinematics of the trial pieces with respect to one another is intended to closely simulate the articulation of the normal knee.

Further, because the nominal kinematics of the prosthesis joint is known, it is not necessary that a large number of images be processed to determine the observed kinematics of the joint, but only a few to correlate with the known kinematics. The observed kinematics of the trial pieces can then be compared by computer 17 to the known kinematics for those trial pieces and anomalies between the kinematics of the trial pieces observed and the nominal kinematics for those trial pieces can be determined. Certain of these anomalies are known to result, for example, from a possible misfit of the trial pieces on the resected bone surfaces. Other anomalies are known to result from an imbalance of the ligaments of the joint. The computer identifies these anomalies and displays suggestions to the surgeon, either via the monitor, the printer or audio speakers, of how to correct such observed anomalies.

In accordance with this invention, with the trial components fitted to the resected knee, as shown in FIGS. 2A and 2B, the surgeon flexes, extends, and rotates the extremity while the trial pieces are within the field of view FOV of camera 11. The camera thus is able to determine the position and orientation of the femoral trial piece relative to the camera and the position and orientation of the tibial trial piece relative to the camera in a series of positions as the knee is flexed, extended and rotated. Computer 17 is then able to transform the positions of the trial pieces relative to camera 11 into a series of views of the femoral trial piece relative to the tibial trial piece such that the kinematics of the trial pieces relative to one another may be determined when the patient's leg (extremity) is flexed and extended. The nominal kinematics of the trial pieces, as installed in a normal knee, is known to computer 17. The kinematics of the trial pieces, as determined by camera 11, is then compared to the kinematics of the trial pieces as installed in a normal knee and any anomalies are determined. Once these anomalies have been determined, computer 17 compares the observed kinematics for the prosthesis components with the known kinematics for those components in a normal knee and suggest to the surgeon which ligaments of the knee may need to be balanced so that the prosthesis components will function correctly as installed in the patient.

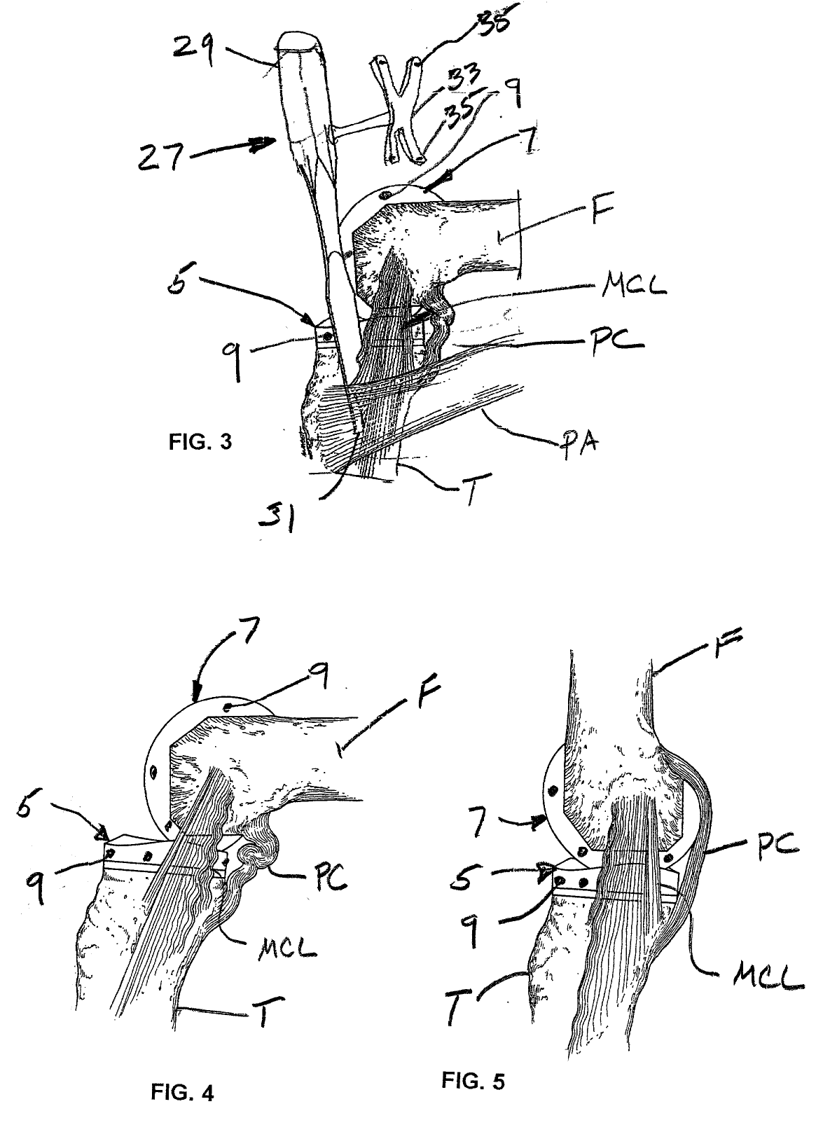

Referring now to FIG. 3, a patient's right knee is illustrated with the femur F and the tibia T resected and with trial components 5 and 7 fitted to the resected surfaces thereof. The knee is shown nearly fully flexed approximately 90° The medial collateral ligament MCL is shown to extend between the proximate end of tibia T and the distal end of femur F. The pes anserinus PA is shown attached to the proximate end of the tibia and the posterior capsule PC on the posterior of the knee is shown to be in a slack position. As shown, the anterior fibers of the medial collateral ligament MCL are in the process of being surgically released by a surgical instrument 27 made in accordance with this invention. Specifically, instrument 27 is like an osteotome having a handle 29 and a sharp blade portion 31. The instrument is provided with a target outrigger 33 which is rigidly affixed to handle 29. The target outrigger serves as a rigid frame for holding four (4) or more targets 35 in known spaced relation to one another and in known spaced relation to blade portion 31. These targets 35 are preferable passive retro-reflective targets similar to targets 9 heretofore described. Alternatively, the targets 35 may be active targets, such as LEDs, which emit light of a known wavelength.

If passive retro-reflectors are used as targets 35, it will be appreciated that in accordance with this invention, the position of targets 35 may be determined by camera 11 and by computer 17 in relation to trial pieces 5 and 7. As will be hereinafter disclosed, once computer 17 determines which ligaments of the knee must be adjusted so as to balance the ligaments of the knee with respect to the prosthetic knee being installed, the camera and the target can determine how much of the ligament and which portion of the ligament should be released or contracted. As the surgeon moves the instrument 27 into the field of view FOV of camera 11, the position and orientation of the instrument with respect to the knee may be determined and as the surgeon performs the procedure to balance the ligaments, as suggested by the camera, the camera and computer can track the amount of adjustment the surgeon has made and can make suggestions as to how much adjustment is still required and the surgeon carries out the procedure.

Referring to FIG. 4, with trial pieces 5 and 7 fitted to the resected surface of the tibia and femur, the posterior portion of the medial collateral ligament MCL is shown to be loose in flexion. From prior testing conducted with the knee in flexion, it has been demonstrated that a knee having an medial collateral ligament MCL in this condition is tight medially in flexion. The algorithm, upon detecting this condition of the medial collateral ligament MCL upon articulation of the knee by the surgeon, would then suggest to the surgeon, as providing a display of suggestions on monitor 21 or aurally over speakers 25, that the anterior portion of the medial collateral ligament is tight and should be relaxed. The surgeon would then take instrument 27 and move it into the field of view FOV of camera 11. The camera would observe targets 35 and would determine the position and orientation of the instrument with respect to trial pieces 5 and 7 and would direct the surgeon to relax the anterior portion of the medial collateral ligament. By observing the distance that the cuffing surface 31 has been moved by the surgeon to release a portion of the medial collateral ligament MCL, the camera and computer can compare the amount of release required to balance the medial collateral ligament with the amount of release actually performed by the surgeon and if additional release is needed, the suggestion can be made by the computer to the surgeon.

FIG. 5 illustrates the knee in extension and illustrates that the posterior fibers of the medial collateral ligament MCL are taut in extension and that the anterior portion of the medial collateral ligament is relaxed. The evaluative tests, as illustrated in FIGS. 2A and 2B, while the knee is in extension demonstrate has normal medial laxity in this position and the medial collateral ligament is abnormally tight and should be relaxed so as to result in better ligament balance with the trial pieces 5 and 7 in place. Again, the computer will suggest to the surgeon the proper procedure for relaxing the medial collateral ligament MCL and will monitor the use of instrument 27 in carrying out this procedure, as described above.

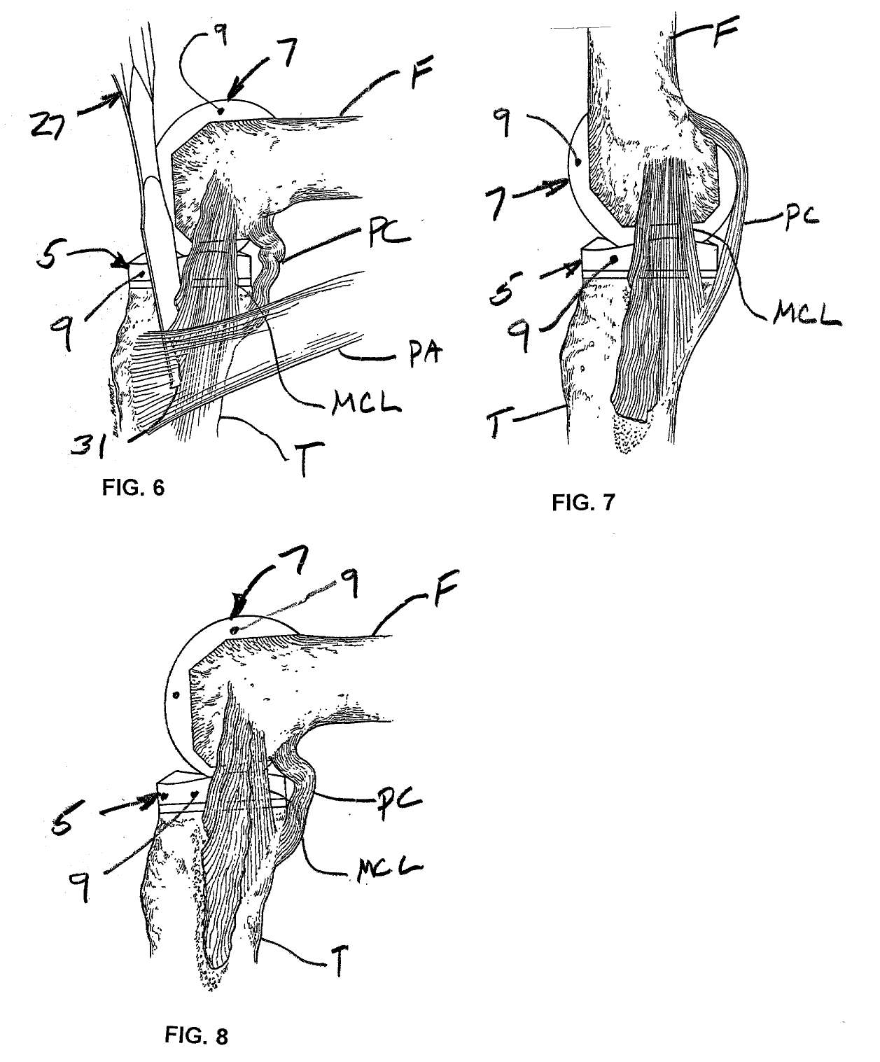

FIG. 6 shows that the anterior fibers of the medial collateral ligament MCL are taut and should be released subperiosteally. These ligament fibers are fairly far distally (e.g., 8-10 cm.), and the osteotome 27 is passed far enough to completely release the anterior fibers. Again, the camera 11 and computer 17 may track the position and orientation of instrument 27 with respect to the trial pieces 5 and 7 and the computer may monitor the release of the medial collateral ligament MCL as determined by the computer. As the tight fibers of the medial collateral ligament MCL are released by the surgeon, the system of the present invention is capable of monitoring the release on the computer generated image of the knee and can suggest to the surgeon when the release is complete or if more of the fibers should be released.

FIG. 7 illustrates that the anterior fibers of the medial collateral ligament MCL have been released. Medial stability in extension is near normal because the posterior portion of the medial collateral ligament MCL and the posterior medial capsule function substantially normally. The tight anterior fibers of the medial collateral ligament MCL have been released from tibia T, and the normal position fibers have been left in tact. Evaluative stability tests performed by the surgeon, as shown in FIGS. 2A and 2B, cause relative movement of the trial pieces 5 and 7 such that no abnormality is observed by camera 11 and computer 17 in extension.

Lastly, referring to FIG. 8, in flexion, the anterior fibers of the medial collateral ligament MCL is shown to no longer to be tight. The posteromedial oblique portion of the medial collateral ligament now acts as a secondary medial stabilizer in flexion. In the flexed position, the released ligaments no longer cause abnormal tightness on the medial side of the knee. The evaluative or evocative tests has shown that restoration of normal laxity of the medial side of the knee has been restored in flexion.

The above examples show how the various evaluative tests performed by the surgeon, as shown in FIGS. 2A and 2B, during the surgery where the trial pieces 5 and 7 have been fitted to the resected knee while the knee is within the field of view FOV of camera 11 can determine anomalies of the knee caused by medial imbalance of the ligaments and how the system of the present invention can suggest surgical procedures to the surgeon to correct this medial imbalance and how the surgical procedures can be monitored as they are being carried out. Of course, many other ligament imbalance conditions can be present. The system of the present invention can detect and suggest corrective procedures for all known ligament imbalance conditions.

It will be appreciated by those skilled in the art that the knee (or any large joint) is complex. However, if a logical plan based on correct alignment throughout the arc of flexion, and on ligament release based on function of each ligament is used, optimum surgical results can be obtained. The ligaments of the knee perform specific known functions, and these functions differ in different position of knee flexion. By programming computer 17 with their known function and with information sufficient to determine whether the ligaments and the trial pieces 5 and 7 are performing optimally as the knee is flexed, both normal and abnormalities of the knee can be determined by camera 11 and computer 17 as the knee is flexed, extended and rotated while being observed by camera 11 during the surgery. These evaluative tests performed by the surgeon during the surgery under the view of camera 11 of the present invention tests the tension or laxity of the ligaments and provides the surgeon with the information necessary to release only the ligaments that are excessively tight, leaving those that are performing normally. This will vary from patient to patient. Fractional ligament release does not destabilize the knee because other ligaments are retained, and because the peripheral attachments of the ligament to other soft tissue structures such as the periosteum or synovial-capsular tissue allow the released ligament to continue to function. Ligament release does not cause instability. It has been found, however, that failure to align the knee properly and failure to release overly tight ligaments does cause instability, unreliable knee function, and excessive wear of the implant components.

In view of the above, it will be seen that the several objects and features of this invention are achieved and other advantageous results attained.

As various changes could be made in the above constructions and methods without departing from the scope of the invention, it is intended that all matter contained in the above description or shown in the accompanying drawings shall be interpreted as illustrative and not in a limiting sense.

What is claimed is:

1. Apparatus for balancing the ligaments of a large joint during joint replacement surgery where a diseased joint is replaced with a prosthesis joint, for comparing the ligaments of the joint undergoing surgery with the ligaments of a normal joint, and for providing the surgeon with information allowing the balancing of the ligaments of the joint after the prosthesis joint has been installed, said apparatus comprising a plurality optical targets mounted on said prosthesis joint fitted to the resected bone surfaces of the joint, one or more video cameras obtaining a series of images of said optical targets as the joint is flexed, extended, and rotated, a computer responsive to the images of the targets to determine the kinematics of the prosthesis joint as it is flexed, extended and rotated, said computer comparing the kinematics of the prosthesis joint with the kinematics of a normal joint and determining, based on anomalies of the observed prosthesis joint relative to the normal joint, whether other prosthesis joint components or spacers are required in order for the prosthesis joint to operate properly and determining which ligaments of the joint must be relaxed or contracted so that the ligaments of the joint are balanced after installation of the prosthesis joint, said computer displaying suggestions to the surgeon for changing said prosthesis components and/or for adding or removing said spacers, and displaying suggestions for relaxing or contracting specific ligaments such that the ligaments of the joint are balanced with the replacement joint in place.

2. Apparatus as set forth in claim 1 wherein said computer determining whether other prosthesis components or spacers are required in order for the prosthesis joint to operate properly and said computer displaying suggestions to the surgeon for changing said prosthesis components or adding or removing spacers.

3. Apparatus as set forth in claim 1 wherein said large joint is a knee, and wherein said apparatus further comprises a trial femoral prosthesis component fitted to the resected surfaces of the femur and a trial tibial prosthesis component fitted to the resected surfaces of the tibia, said trial prosthesis components having said optical targets affixed thereto.

4. Apparatus for determining the kinematics of a large joint undergoing arthroplasty, for comparing the kinematics of the joint with trial prosthetic joint components fitted to resected bone surfaces of the joint with the kinematics of a normal joint, and for providing the surgeon with information allowing the balancing of the ligaments of the joint with a prosthesis joint in place, said apparatus including a plurality of optical targets mounted on said trial joint replacement components fitted to the resected bone surfaces of the joint, the position and orientation of each of said targets being known with respect to its respective said trial component such that by knowing the position and orientation of said targets of said respective trial components the position and orientation of said trial components is known, one or more video cameras, said one or more cameras being positioned with respect to said joint such that as the joint is flexed, extended and rotated by the surgeon said targets are within the field of view of said one or more cameras, said one or more cameras obtaining a series of images of the optical targets as the joint is flexed, extended, and rotated, a computer responsive to the images of the targets for determining the position and orientation of the targets and hence of the trial components with respect to said one or more cameras, said computer determining the position and orientation of said trial components with respect to one another as the knee is flexed, extended and rotated thereby enabling the determination of the kinematics of the joint, said computer comparing the kinematics of the observed joint with the kinematics of a normal joint and determining, based on anomalies of the observed joint relative to the normal joint, which ligaments must be relaxed or contracted so that with the prosthesis joint installed the ligaments are balanced, said computer displaying suggestions to the surgeon for relaxing or contracting specific ligaments so that the ligaments may be balanced.

5. Apparatus for determining the kinematics of prosthesis joint components relative to one another and relative to a normal joint during joint replacement surgery to aid the surgeon in fitting joint components to the resected bones of the joint undergoing surgery and to aid the surgeon in balancing the ligaments of the joint after a prosthesis joint has been installed, said apparatus comprising:
a. a first set of optical targets mounted to a first trial joint component fitted to a first resected bone of the joint, said first set of optical targets being in a predetermined geometrical relationship with respect to said first trial joint component;
b. a second set of optical targets mounted to a second trial joint component fitted to a second resected bone of the joint, said second set of optical targets being in a predetermined geometrical position with respect to said second trial joint component;
c. a video camera disposed to receive images of said first optical targets and said second optical targets, said video camera having a field of view, said first and second sets of targets being within said field of view as the joint is flexed, extended and rotated;
d. a computer operatively connected to said camera, said computer being responsive to the images of said first and second sets of targets to determine values of the position and orientation of said trial joint replacement components in space and with respect to one another and to determine the kinematics of the trial joint components relative to one another as the joint is extended, flexed and rotated; and
e. said computer displaying information to the surgeon relating to the balancing of the ligaments of the joint.

6. A method of balancing the ligaments of the knee during total knee arthroplasty comprising the steps of:
a. resecting the distal end of the femur so as to receive a femoral prosthesis implant;
b. resecting the proximal end of the tibia so as to receive a tibial prosthesis implant:
c. fitting trial implant components to the resected ends of the femur and tibia, said trial components having a plurality of optical targets affixed thereto with the location of the targets for each trial component being known;
d. manipulating the extremity in flexion, extension and rotation;
e. viewing said optical targets on said trial components with an video camera as said extremity is manipulated;
f. determining the position and orientation of the targets with respect to said camera;
g. determining the position and orientation of said trial components with respect to one another so that the kinematics of the trial components relative to one another may be determined;
h. identifying anomalies between the observed kinematics of the trial components as the extremity is manipulated which result from an unbalance of the ligaments of the knee; and
i. suggesting to the surgeon which of the ligaments of the knee should be adjusted either by relaxing or contracting such ligaments such that upon installation of the prosthesis components the ligaments of the knee are balanced.
